(19) United States
(12) Patent Application Publication
(10) Pub. No.: US 2003/0017552 A1
(43) Pub. Date: Jan. 23, 2003

(54) Modular vector systems

(76) Inventors: Kevin A. Jarrell, Lincoln, MA (US); William Donahue, Quincy, MA (US)

Correspondence Address: Choate, Hall & Stewart, Exchange Place, 53 State Street, Boston, MA, 02109, US

(21) Appl. No.: 09/910,354

(22) Filed: Jul. 20, 2001

Related U.S. Application Data
(60) Provisional application No. 60/219,820, filed on Jul. 21, 2000.

(51) Int. Cl. C12P 19/34; C12N 15/85; C12N 15/74

(52) U.S. Cl. 435/91.2; 435/455; 435/320.1

(57) ABSTRACT

The present invention encompasses the recognition that vectors utilized in molecular biology need not be provided as single intact molecules but rather can be assembled from fragments containing functional elements, or portions thereof. In certain preferred embodiments of the invention, vector fragments are linked together simultaneously with the linkage of vector and insert sequences to one another.

### PRIORITY CLAIM AND RELATED APPLICATIONS

The present application claims priority under 35 USC § 119 to U.S. Ser. No. 60/219,820, filed Jul. 21, 2000, the entire contents of which are incorporated herein by reference. The present application is also related to co-pending applications U.S. Ser. No. 09/225,990, filed Jan. 5, 1999 and U.S. Ser. No. 09/897,712, filed Jun. 29, 2001, the latter being a nationalized application corresponding to PCT/US00/001 89, filed Jan. 5, 2000; the entire contents of each of these applications are incorporated herein by reference.

### GOVERNMENT FUNDING

Some or all of the work described herein was supported by grant number MCB9604458 from the National Science Foundation; the Unites States Government may have certain rights in the invention.

### BACKGROUND OF THE INVENTION

Perhaps the classic genetic manipulation in molecular biology is the cleavage of a circular vector with one or more restriction enzymes and the ligation of a selected insert into the linearized vector. Since the 1970s, when the pioneers of molecular biology first demonstrated such manipulation to be feasible, significant research effort has been invested in the development of improved vector systems (see discussion of vectors derived from plasmids in Ausubel et al., Current Protocols in Molecular Biology, Section II, 1.5.1-1.5.17, John Wiley & Sons, 1998, incorporated herein by reference).

To give but a few examples, plasmid vectors that replicate in different hosts, with different copy numbers, have been prepared (e.g., bacterial vectors designed to have either relaxed or stringent control of replication; yeast vectors with either a 2μ or centromeric replication origin, mammalian vectors containing viral [e.g., SV40 or BPV] origins of replication, etc.). Vectors have been engineered to allow ready detection of insertion events (e.g., by creation or disruption of a selectable or detectable marker), to direct high levels of expression of proteins encoded by inserted sequences (e.g., under the control of transcription, splicing, and/or translation signals active in a given host system), to generate gene fusions that allow analysis of expression of inserted sequences (e.g., by analysis of B-galactosidase, chloramphenicol transferase, luciferase, or green fluorescent protein activity, etc.), or to create fusion proteins with experimentally useful attributes (e.g., easy purification, desired cellular localization, etc.). Vectors have been designed that are particularly useful for determining the sequence of inserted fragments (e.g., by allowing easy production of single-stranded DNA), or for producing RNA (sense or antisense) from the inserted sequences. Most companies that sell molecular biology reagents include among their products vectors that they have developed to be particularly useful for designated applications (see, for example, catalogs provided by Amersham Pharmacia Biotech, Piscataway, N.J.; Promega Corporation, Madison, Wis.; Invitrogen Inc., Carlsbad, Calif.; Life Technologies, Inc., Rockville, Md.; New England Biolabs, Beverly, Mass.; Stratagene, Inc., La Jolla, Calif.).

Of course, the universe of genetic “vectors” is not limited to circular molecules derived from bacterial plasmids. Any nucleic acid molecule that includes sequences sufficient to direct in vivo or in vitro self-replication can be employed as a vector. Typically, such replication sequences include a replication origin that directs duplication of the vector sequence in a host system (typically a transformed cell). Alternatively, sequences that direct integration of the vector into another nucleic acid molecule that is present in and replicated by the relevant host system can be sufficient to achieve vector (and insert) replication.

Most vectors in use today are derived from naturally-occurring bacterial plasmids, bacteriophages, or other viruses. Some vectors contain features of more than one of these systems. Almost all of the commonly-used vectors contain one or more restriction sites designed for convenient insertion of fragments; most have at least one polylinker (see, for example, the vector database maintained at http://vectorbd.atcg.com/vectordb/vector.html, the contents of which as of Jul. 19, 2000 are included herein as Appendix A).

Despite the broad availability of vectors from commercial and other sources, each one has features selected by the relevant manufacturer rather than the experimental user. It is not uncommon for a researcher to have to modify an available vector to suit his experimental needs, or alternatively to modify his experimental design to accommodate the available vectors. There remains a need for the development of techniques and reagents that would allow a researcher to readily design and assemble vector(s) appropriate to his experimental needs.

### SUMMARY OF THE INVENTION

The present invention encompasses the recognition that vectors are comprised of modular elements and need not be provided as discrete nucleic acid molecules into which fragments of interest are inserted. Rather, vectors can themselves be assembled from pieces that contain part or all of individual useful elements. In certain preferred embodiments of the invention, fragments corresponding to pieces of what is traditionally viewed as the “vector backbone” are provided individually and are linked to one another substantially simultaneously with the linkage that associates vector sequences with insert sequences.

According to the present invention, components of a vector can be defined as one of a variety of categories of vector elements. For example, sequences that allow the vector to replicate in a host system may be classified as “replication elements”. Similarly, sequences that allow host cells containing a vector to survive experimental conditions that kill otherwise identical host cells lacking a vector may be classified as “replication elements”; sequences that allow detection but not selection of host cells containing vector sequences, or host cells containing vector and insert sequences, may be classified as “detectable elements”; sequences that can act to direct expression (i.e., transcription, splicing, and/or translation) of other sequences can be classified as “expression elements”. Other categories of elements may also be defined as discussed in further detail herein.

The present invention allows a researcher to select individual elements from one or more categories of vector elements, and to combine the selected element(s) with one or more individual element(s) with one another to assemble vectors that contain a desired collection and arrangement of elements. Individual vector elements, or portions or combinations thereof, are provided on separate “vector fragments” that are linked together to create the final vector. Thus, the present invention provides techniques and reagents useful in the assembly of vectors from individual vector fragments. Preferably, a vector assembled according to the present invention will include at least a replication element. More preferably, the vector will include one or more additional elements selected from the group consisting of additional replication elements (e.g., effective in different host systems), selectable markers, detectable markers, expression elements, fusion protein elements, mobile elements, recombination elements, cleavage site elements, etc. The inventive techniques and reagents may be employed to link two or more vector fragments to one another, serially or simultaneously, and also to link vector fragments with one or more insert fragments (again, serially or simultaneously).

In particularly preferred embodiments of the present invention, one or more of the vector and insert fragments used in the assembly of a final hybrid construct is prepared without the use of restriction enzymes (or any endonuclease). Most preferably, substantially all of the fragments that become linked together to produce a final assembled molecule are prepared without the use of restriction enzymes. In particularly preferred embodiments of the invention, RNA-Overhang Cloning and/or DNA Overhang Cloning are employed to produce vector and/or insert fragments. Also, in certain preferred embodiments of the invention, vector fragments, and optionally insert fragments, are linked to one another by ligation-independent cloning (i.e., without the use of a ligase enzyme).

### DESCRIPTION OF THE DRAWING

FIG. 1 depicts assembly of a hybrid molecule comprising λ vector elements and an insert, according to the present invention.

FIG. 2 shows assembly of a hybrid molecule comprising bacterial vector elements and an insert in a three-molecule linkage reaction according to the present invention.

FIG. 3 depicts assembly of a hybrid molecule containing bacterial vector elements and an insert according to the present invention. Two vector fragments and one insert fragment are linked together to form a hybrid that can be selected by growth in the presence of tetracycline and lack of growth in the presence of ampicillin.

FIG. 4 depicts assembly of a hybrid molecule comprising bacterial vector elements and an insert according to the present invention. Two vector fragments, each of which contains a portion of a detectable element, and one insert fragment are linked together to form a hybrid. Hybrids that contain insert can be distinguished from those that do not by a blue/white screen.

FIG. 5 shows assembly of a hybrid molecule containing bacterial vector elements and an insert according to the present invention. Two vector fragments, one of which contains a bacterial origin of replication and a first portion of a LacZ gene and one of which contains an ampicillin resistance gene and a second portion of the LacZ gene are linked to an insert fragment. Hybrids can be selected by growth in the presence of ampicillin; those containing insert can be distinguished from those lacking insert by a clue/white screen.

FIG. 6 shows assembly of a hybrid molecule from three vector fragments and one insert fragment. Linkage of the four fragments re-creates two vector elements, and operatively links a third (the promoter) with the insert sequences.

FIG. 7 shows collections of vector fragments, each of which contains only a single vector element, that may alternatively be linked to each other and an insert to form a hybrid molecule according to the present invention.

FIG. 8 depicts a kit comprising two collections of vector fragments that can be used in various combinations to create vectors with different attributes according to the present invention. The first collection of vector fragments contains three fragments, each of which includes the pGal promoter and a first portion of a selectable marker selected from the group consisting of the URA3, TRP1, and HIS3 genes. The second collection of vector fragments contains six different fragments, each of which contains a second portion of one of the selectable markers, and an origin of replication that is either a centromeric origin or a 2μ origin.

FIG. 9 depicts assembly of a hybrid molecule from two vector fragments and one insert fragment, each of which was prepared by DOC, according to the present invention. Panel A shows the generation of the two vector fragments; Panel B depicts the ligation of these two fragments with the insert fragment to produce the final hybrid.

FIG. 10 shows a hybrid molecule assembled from two vector fragments and are insert fragment, each of which was prepared by DOC, according to the present invention.

FIG. 11 shows the primers used

(3NT5′OST [SEQ ID NO:_]; 3NT3′OHT [SEQ ID NO:_];
3NT5′KHT [SEQ ID NO:_]; 3NT3′KST [SEQ ID NO:_];
1NT5′OSI [SEQ ID NO:_]; 1NT3′Ori(s) [SEQ ID NO:6];
1NT5′KAN [SEQ ID NO:11]; 1NT3′KAN [SEQ ID NO:12].

### DEFINITIONS

“Element”—The term “element” is used herein to refer to a region of nucleic acid sequence that imparts a particular functional or structural characteristic upon the molecule.

“Expression”—“Expression” of a nucleic acid sequence, as that term is used herein, refers to one or more of the following events: (a) production of an RNA template from a DNA sequence (e.g., by transcription); (b) processing of an RNA transcript (e.g., by splicing, editing, and/or 3′ end formation); (c) translation of an RNA has been into a polypeptide or protein; (d) post-translational modification of a polypeptide or protein.

“Fragment”—A “fragment”, as that term is used herein, is an individual nucleic acid molecule that can be hybridized or linked with one or more other fragment molecules to produce a hybrid molecule. Preferably, a fragment contains at least a portion of a selected sequence element so that, when the fragments are linked together, a hybrid molecule is generated that contains a predetermined collection and arrangement of sequence elements. In certain preferred embodiments of the invention, each fragment contains at least one intact sequence element. In other preferred embodiments, each fragment contains only one intact sequence element. In still other preferred embodiments, at least one fragment contains only a portion of a particular sequence element (though the fragment may also contain a complete copy of a different sequence element). Preferably, that fragment will become linked with another fragment so that the complete sequence element is reassembled in the final hybrid. Alternatively or additionally, fragments are selected so that different hybrid molecules can be produced from linkage of the same collection of fragments, and such different hybrids can be distinguished from one another on the basis of whether a particular sequence element is recreated in the hybrid. Preferred fragments for use in accordance with the present invention are prepared without the use of restriction enzymes. Most preferably they are prepared by polymerase chain reaction (PCR) amplification according to ROC or DOC techniques (see, for example, U.S. Ser. No. 60/114,909, U.S. Ser. No. 09/225,990, and Coljee et al., Nature Biotechnology 18:789, July 2000, each of which is incorporated herein by reference in its entirety). Preferred fragments are double stranded nucleic acid molecules with at least one single-stranded overhang.

“Host system”—A “host system” according to the present invention is any in vivo or in vitro system into which a vector is introduced. Preferably, the host system is a cell or organism. Any type of cell, including a bacterial cell, yeast cell, plant cell, or animal cell, can be a host cell. Cells in culture and cells that are part of living tissues or organisms can also be host cells.

“Hybrid”—A “hybrid” nucleic acid molecule according to the present invention is a molecule produced by hybridization and/or linkage of at least two fragments or elements to one another.

“Linkage”—The “linkage” of two or more nucleic acid molecules to one another according to the present invention refers to any reaction that results in formation of a covalent bond between two nucleic acid molecules that were not covalently attached to one another prior to the linkage reaction. Preferably, the linkage is accomplished either by splicing or by ligation. Alternatively, linkage may be accomplished indirectly, for example by replication of molecule pairs (or clusters) held together by ligation but including one or more nicks. Linkage may occur in vitro or in vivo.

“Overhang”—An “overhang”, according to the present invention, is a single-stranded region of nucleic acid extending from a double-stranded region. Preferred overhangs are at least one nucleotide long. Particularly preferred overhangs are at least 2, 3, 4, 5, 5, 6, 7, 8, 9, 10, 11, 12, 13, 14, 15, 20, or 25 nucleotides long. In some preferred embodiments of the invention, the overhangs are comprised of at least one, preferably at least 2, 3, 4, 5, or more RNA residues; in other preferred embodiments the overhangs are comprised of DNA. In some embodiments of the invention, overhangs may comprise RNA elements that include functional intronic sequences.

“Portion”—A “portion” of a nucleic acid molecule or polypeptide molecule, as that term is used herein, is any piece that is shorter in length than the entire molecule. Preferably, a portion has a length sufficient to be characteristic of the full length molecule. For nucleic acid molecules, preferred portions are usually at least about 3-5 residues in length, more preferably at least about 5, 10, 15, 20, 25, 30, 35, 40, 45, 50, or I100 residues in length. For polypeptide molecules, preferred portions are typically at least about 2-5 residues in length, more preferably at least about 7, 10, 15, 20, 25, 30, or 40 residues in length.

“Primer”—The term “primer”, as used herein, refers to a polynucleotide molecule that is characterized by an ability to be extended against a template nucleic acid stand, so that a polynucleotide strand whose sequence is complementary to that of at least a portion of the template strand, is produced linked to the primer. Preferred primers are at least approximately 5-10 nt long; particularly preferred primers are at least about 15 nt long. In many preferred embodiments, primers preferably have a length within the range of about 18-30 nt, preferably longer than approximately 20 nt.

### Description of Certain Preferred Embodiments of the Invention

As described above, the present invention recognizes that vectors need not be provided as intact, discrete molecules, but rather can be provided as fragments that contain all or part of particular desired sequence elements. The invention provides techniques and reagents for the assembly of vectors (and/or inserts) through the linkage of such fragments. Certain preferred embodiments of this invention are described in more detail below.

Vector Elements

As will be appreciated by those of ordinary skill in the art, any desired nucleic acid sequence can be considered a vector element according to the present invention. Practitioners will be aware of their own needs and desires in terms of vector functions and attributes, and will readily be able to select appropriate sequences for use as vector elements. Nonetheless, certain types of sequence elements are already well established as useful in the field of vector construction. For example, Invitrogen Corporation, one of the larger distributors of molecular biology reagents, provides on its web site (www.invitrogen.com) a page entitled “Anatomy of a Vector” that lists the following categories of vector elements: promoters, inducible elements, transcriptional termination sequences, origins of DNA replication, affinity purification tags, multiple cloning sites/polylinkers, and selectable markers. The contents of this site, as they were presented on Jul. 19, 2000, are included herein as Appendix B.

Replication Elements

As described above, any sequence that operates to ensure replication of vector sequences in a selected host system constitutes a replication element. A variety of replication elements are already available in the art, and have been employed in commonly-available vector systems (see, for example, Ausubel et al., Current Protocols in Molecular Biology, Section II, Unit 1.5.1-1.5.17, John Wiley & Sons, 1998, the entire contents of which are incorporated herein by reference).

It will be appreciated by those of ordinary skill in the art that it is often desirable to construct a vector containing more than one replication element. For example, if it is desired that the same vector be able to replicate in more than one host cell type (e.g., in both bacterial cells and mammalian cells), then the vector should be designed to include replication elements that operate in each relevant cell type. On the other hand, it is also known that certain replication elements are incompatible with one another in a given cell type. It is generally desirable not to include incompatible elements in a single construct unless fragmentation of the construct in the host cell is desired.

Available replication elements that are known to operate in E. coli, the most commonly employed bacterium in molecular biology, include both high copy (so-called “relaxed control”) elements such as pMBI (100-300 copies/cell; Bolivar et al., Gene 2:95, 1977), ColE1 (>15 copies/cell; Kahn et al., Method. Enzymol. 68:268, 1979) and p15A (about 15 copies/cell; Chang et al., J Bacteriol. 134:1141, 1978) and low copy (so-called “stringent control”) elements such as pSC 101 (about 6 copies/cell; Stoker et al., Gene 18:335, 1982), F (1 to 2 copies/cell; Kahn et al., Method. Enzymol. 68:268, 1979), and RK2 (2-4 copies/cell; Kahn et al., Method. Enzymol. 68:268, 1979). The RI (low copy at 30° C. and high copy above 35° C.; Uhlin et al., Gene 22:225, 1983) replicon also operates in E. coli, as do various phage origins of replication including λ dv (Jackson et al., Proc. Natl. Acad. Sci. USA 69:2904,1972), m13, f1, etc.

Replication elements that are known to operate in bacteria other than E. coli include RK2 and RSF1010, which have been shown, unlike ColE1, to have relatively broad host-ranges. In some cases, it may be desirable (or necessary) to introduce vectors into bacterial host cells through a mating process, in which case sequence elements encoding certain trans-acting factors (e.g., the tra or mob genes) may be required, as may be the cis-acting oriT site.

There are two primary categories of replication elements known to operate in yeast cells, centromeres and the 2μ replicon. Of course, since DNA can readily be targeted for integration in yeast cells, it is not always necessary for a vector to be used in yeast cells to include an origin of replication that is active in those cells. Sequences that target integration of the vector into other replicating nucleic acid molecules are sufficient to constitute a replication element according to the present invention in those circumstances.

Several viral origins of replication, such as simian virus 40 [SV40], bovine papilloma virus [BPV], and Epstein Barr Virus [EBV], oris are known to operate in mammalian cells (sometimes requiring the presence of additional viral genes) and therefore can be employed as mammalian replication elements according to the present invention. Alternatively, sequences sufficient to target integration of a vector into another nucleic acid molecule (e.g., a chromosome or virus) capable of replicating in the mammalian cell can be employed. Targeted homologous recombination has been demonstrated to work effectively in mammalian cells, so that regions of homologous gene sequence can operate as replication elements according to the invention. Analogously, sequence elements of the Cre recombinase system can be employed to direct integration of vector sequences in mammalian systems (see, for example, Fukushige et al., Proc. Natl. Acad. Sci. USA, 1992).

Viral origins of replication such as the baculovirus origin are known to operate in insect cells and can be employed as replication elements according to the present invention, as can other sequences, such as P-element sequences, that enable integration of vector sequences into other replication-competent nucleic acids.

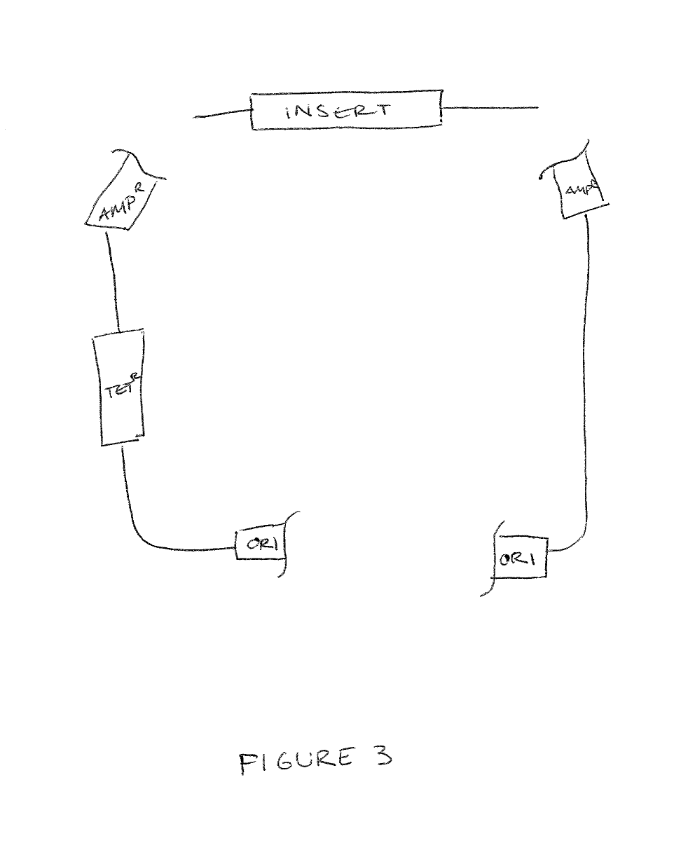

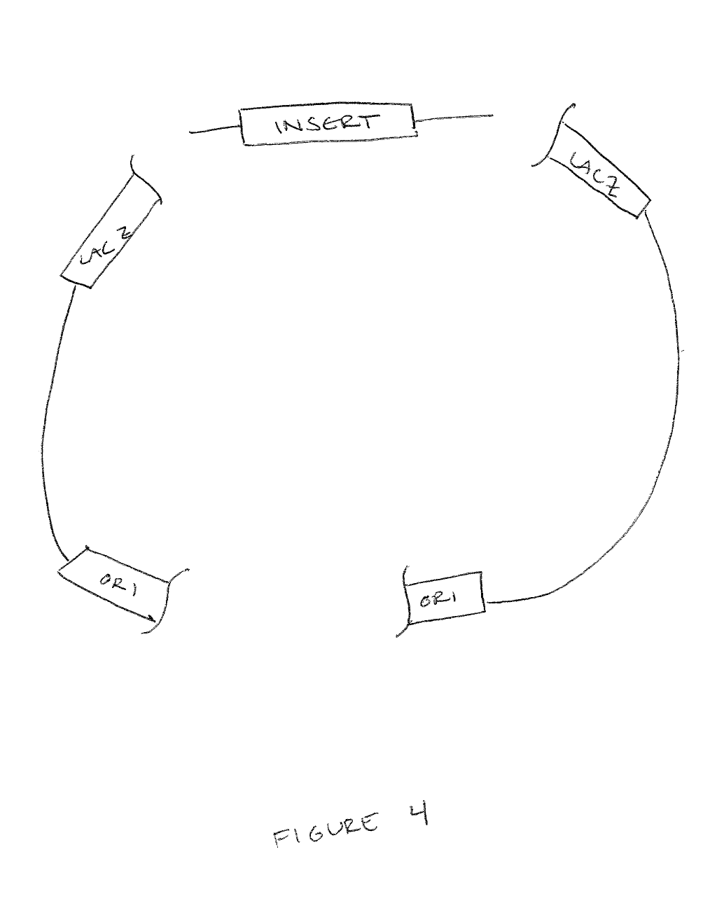

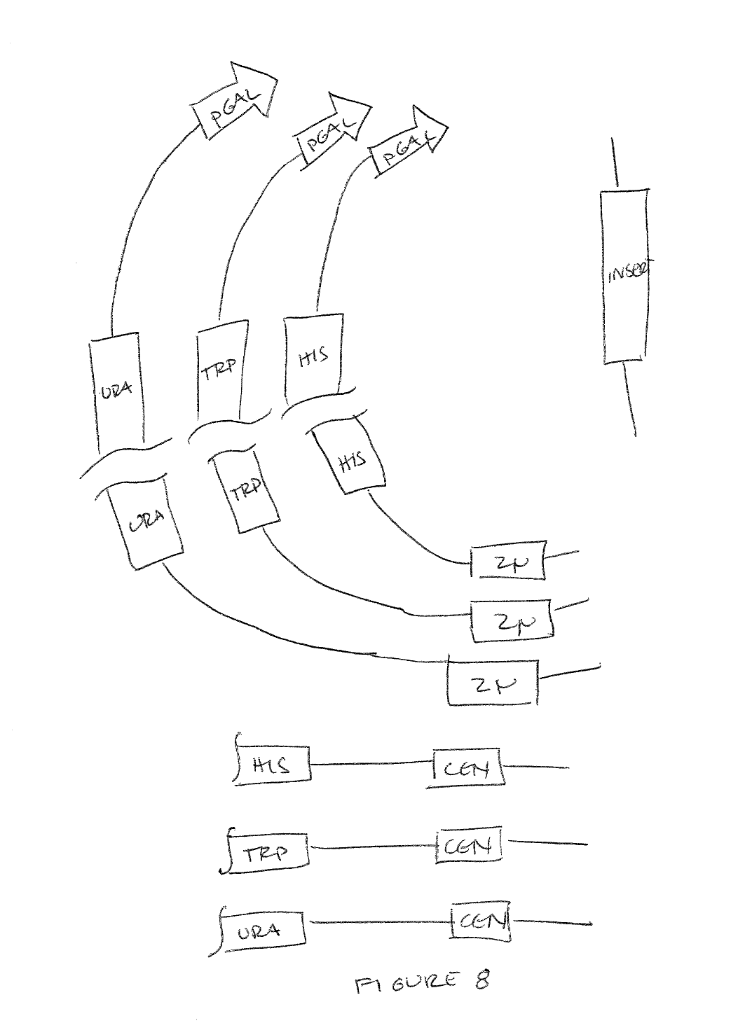

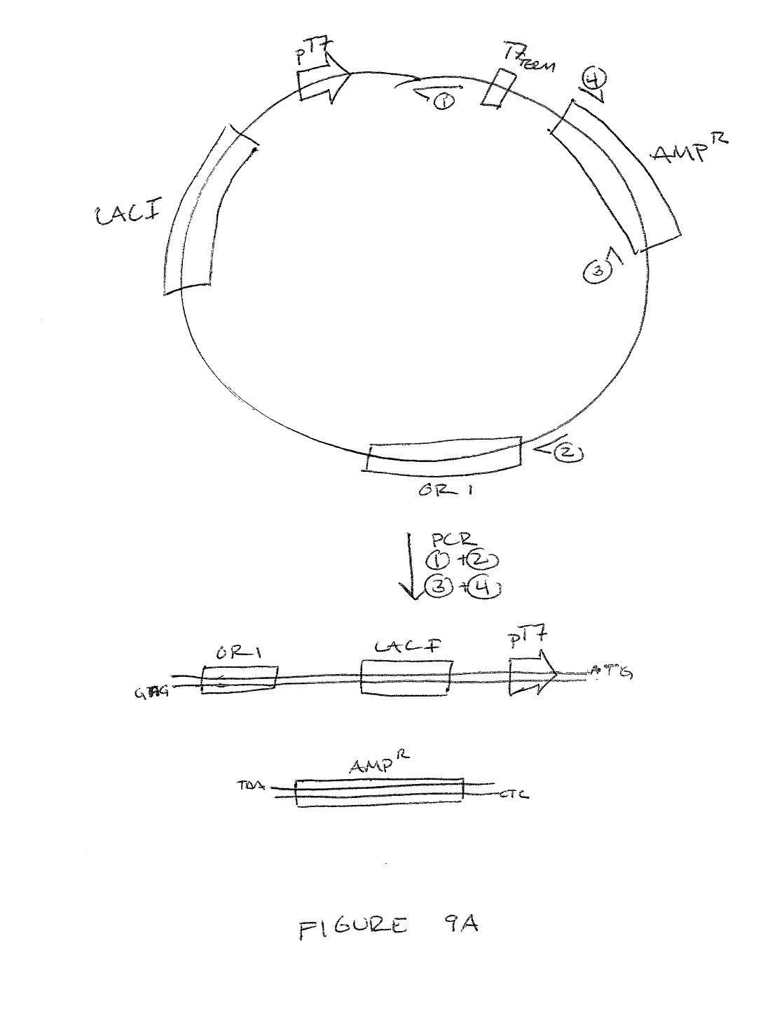

In certain embodiments of the invention, it will be desirable to provide a particular replication element in two parts, on two different fragments, so that hybrid molecules will only replicate if they contain properly ligated fragments (see, for example, FIGS. 3, 4, and 6). In other embodiments, replication elements are provided intact on a single vector fragment (see, for example, FIGS. 2, 5, and 7-9).

Vector Detection Elements

A wide variety of sequences are available that allow host cells containing vector to be distinguished from host cells that do not contain vector. There are two basic categories of such elements: those that contain a selectable marker (i.e., one that imparts a growth advantage to vector-containing cells under certain conditions) and those that contain a detectable marker. A wide variety of such markers is available, for use in different cell types.

The most commonly employed selectable markers utilized in bacterial systems are those that confer resistance to antibiotics such as ampicillin, chloramphenicol, kanamycin, and tetracyline. Similarly, selectable markers commonly utilized in insect and/or mammalian cells include those that confer resistance to zeocin, neomycin, blasticidin, or hygomycin. The DHFR gene, which confers the ability to grow in the absence of exogenous purines (and also confers resistance to methotrexate, can also be used as a selectable marker in a range of cell types including mammalian cells. Also, cytosine deaminase can be used as a selectable marker under conditions that require cells to convert cytosine to uracil for growth. Other selectable markers useful in mammalian cells include, for example, hygromycin-β-phosphotransferase (HPH), puromycin-N-acetyl transferase (PAC), thymidine kinase (TK), and xanthine-guanine phosphoriboseultransferase (XGPRT).

The most commonly employed selectable markers utilized in yeast cells include those that confer the ability to grow in the absence of a given nutrient such as uracil, tryptophan, histidine, leucine, lysine, etc.

Preferred detectable markers for use in accordance with the present invention include genes encoding proteins that produce detectable products. Commonly employed detectable markers include, for example, the β-galactosidase gene, the green fluorescence protein gene, the horse radish peroxidase gene, the nitric oxide syntheses gene, the chloramphenicol acetyl transferase gene, the luciferase gene, etc.

Those of ordinary skill in the art will readily appreciate that most or all of these vector detection elements can alternatively be employed as insert detection elements. For example, FIGS. 3-5 depict inventive reactions in which vector fragments are designed so that, if they become linked to one another, a vector detection element is created. On the other hand, if an insert fragment becomes linked between them, the vector detection element is not created. Thus, constructs containing the insert fragment and those not containing the fragment can readily be distinguished from one another.

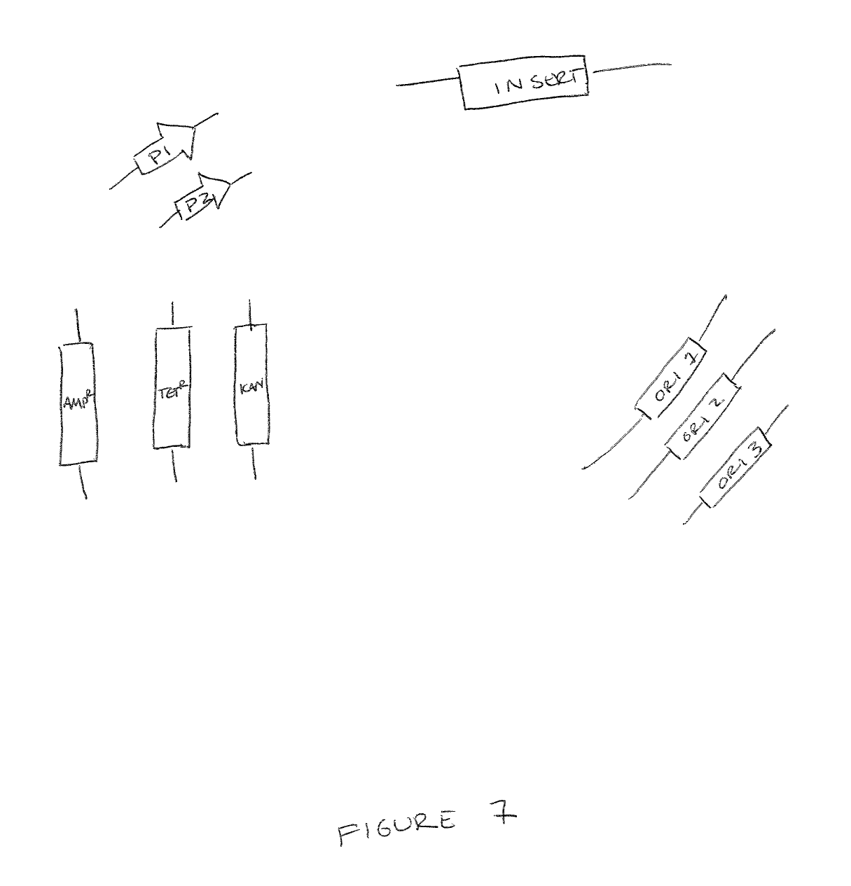

Similarly, those of ordinary skill in the art will appreciate that it will often be desirable to design vector and/or insert fragments so that a vector detection element is only created if the fragments become linked together in the desired arrangement. FIG. 6, for example, depicts a particular embodiment of the invention in which this strategy was employed to simplify hybrid construct production according to the present invention.

It should be noted that one advantage of the present invention is that it renders the insert detection strategies described in the previous two paragraphs particularly practicable. The inventive modular approach to vector assembly, and particularly the inventive employment of cloning technologies that do not require restriction digestion, removes the need for a polylinker in order to introduce insert sequences into a vector. Since polylinkers add unnatural sequences, their location in the middle of a detectable or selectable gene typically disrupted the gene activity, so that it was not possible to use reverse selection or detection to assay for insert insertion. By contrast, the inventive technologies allow the seamless union of insert and vector sequences, making feasible the use of these convenient screens and selections.

Expression Elements

As will be appreciated by those of ordinary skill in the art, one of the most common uses of vector systems in molecular biology is to arrange for expression of insert sequences in a host cell of interest. Any sequence that participates in directing or regulating expression of a linked sequence can be an expression element according to the present invention. A wide variety of such sequences are known in the art; certain examples are discussed in more detail below.

PROMOTER: Promoters are the regions of DNA that are responsible for establishing the initiation site for transcription. A variety of different promoters, operative in different systems, have been defined and characterized. Different promoters may direct expression of linked sequences at different levels. Furthermore, some promoters are constitutively active, while others can have their activity modulated through adjustment of the experimental conditions. Some promoters are active in only particular cell types, where as others are ubiquitously expressed.

Preferred promoters known to be active in bacterial cells include, for example, P_{BAD}, P_{L}, P_{R}, lack, tack, trc, spa lacUV5, T3, T7, T7 LAC, SP6, etc.; preferred promoters known to be active in yeast cells include, for example pGAL1, pAOX1, pADH, etc.; preferred promoters known to be active in insect cells include, for example, the MT, Ac5, and polyhedrin promoters, etc; preferred promoters known to be active in mammalian cells include, for example, P_{ΔHSP}, P_{SG}, P_{CMV}, P_{EF-1α}, P_{SV40}, P_{RSV}, P_{PGK}, P_{MMTV, P}_{MC1} etc.

ENHANCERS/TRANSCRIPTIONAL REGULATORS: Regulator sequences that operate to stimulate or repress transcription from a given promoter in certain cell types or under certain conditions can often be combined with any of a variety of different promoters to create a transcription control element with useful characteristic. The universe of known regulatory sequences operative in different organisms is very large. Particularly preferred elements that are commonly used in vector systems include, for example, the lac operon, the λ cI site, the tet operon, lexA sites, Gal4 sites, the SV40 enhancer, the MMTV enhancer, etc. Those of ordinary skill in the art will immediately recognize the huge range of alternative sequences that could be employed in the practice of the present invention. Experiments to define additional such sequences, operative in the context of any particular experiment, are routine.

TRANSCRIPTION TERMINATOR: Although not required, it is sometimes desirable to include in an expression vector sequences that will terminate transcription of relevant sequences at a selected point. Without such termination signals, it may be possible for RNA polymerases, at least under some circumstances, to transcribe indefinitely around a circular construct. A variety of different transcriptional termination sequences have been identified; the one most commonly used in vector applications is probably the SV40 terminator. Alternatively or additionally, 3′-end formation signals, such as polyadenylation sites, may be employed.

SPLICING SIGNALS: In certain circumstances, it may be desirable to include in inventive expression vectors signals that can direct splicing of transcripts encoded by insert sequences. For example, if a vector includes a promoter and exonic sequences including a splice donor site, then insert sequences containing a splice acceptor site can be expressed and translated. In certain embodiments of the invention, it might be desirable to provide a collection of vectors or vector fragments (3) that contain the splice acceptor site in all three possible frames, so as to ensure in-frame fusions of insert sequences in one version of the vector, regardless of whether information about the insert sequence is available.

TRANSLATION START: Often, if expression of an insert that does not include 5′ sequences (or is not known to include such sequences) is desired, it will be useful to include translation start sequences. The consensus translation start sequence, known as the Kozak sequence, will provide the strongest translation initiation signal, but in most cases a single ATG reasonably positioned with respect to the start of the transcript will suffice.

TRANSLATION STOP: Expression vectors designed to express insert sequences that may be lacking their natural 3′ ends often benefit from the inclusion of translation stop sequences. As with the translation start and splicing sequences, families of vectors can be prepared containing the relevant sequences in all three possible frames so that knowledge of the insert sequence is not required. Alternatively, a single vector could be employed but families of insert fragments can be prepared with additional (or fewer) nucleotides on one or both ends.

Gene Fusions

As those of ordinary skill in the art will be aware, a variety of vector systems have been engineered to generate gene fusions between insert sequences and a reporter gene in the vector backbone. Such fusions are useful, for example, to detect expression patterns of the insert sequences, or to detect expression control elements that may be present in the insert sequences. Gene fusions may also allow a researcher to track the expression products of the fused gene.

Particularly preferred detectable genes for use in gene fusion applications include, for example, LacZ, chloramphenicol acetyl transferase (CAT), green fluorescence protein (GFP), luciferase, horse radish peroxidase (HRP), etc.

Fusion Proteins

One version of gene fusions that is particularly commonly employed in vector systems is fusions that generate fusion proteins with a desirable characteristic. As will be appreciated, it will often be desirable to provide families of vectors or vector fragments that allow C-terminal, N-terminal, or internal fusions, and also that allow fusions in all possible frames, preferably without knowledge of insert sequence.

For example, a variety of sequence elements are available that encode polypeptides that, when fused to a polypeptide encoded by an insert sequence, allow that polypeptide to be readily purified. Particularly preferred purification tags include, for example, (His)₆, thioredoxin, glutathione-S-transferase, streptavidin, staphylococcal protein A (which interacts strongly with IgG; Amersham Pharmacia Biotech, Piscataway, N.J.), etc.

Also available are a variety of sequence elements encoding detectable moieties, such as epitopes for which high-specificity antibodies are available, that can be useful in the detection of an expression fusion protein. Examples of such detectable epitopes include, for example, Xpress™, c-myc, CA25, thioredoxin, V5, HA, calmodulin binding peptide (CBP), Aag, etc.

In some cases, it is desirable to remove the protein tags created by fusion of encoding insert sequences with encoding vector sequences. Sequence elements encoding polypeptide cleavage elements (e.g., by furin, enterokinase, thrombin, factor X1, PreScission, etc.) are particularly useful in such applications.

Other useful sequence elements for the production of fusion proteins are ones that encode targeting moieties, such as secretion signals (e.g., BiP for insect cells, human placental alkaline phosphatase or human growth hormone for mammalian cells, protein A for bacterial cells, etc.) or other elements, that direct the fusion product to a particular cellular location. Examples of such targeting sequences include, for instance, yeast AgA2 sequences that target the fusion protein to the cell surface, VP22 fusions that target to the mammalian nucleus, pRLT3-NLS, COXVIII signal, etc.

Polylinkers

One virtually ubiquitous element in most commercially-available vectors today is a so-called “polylinker” or “multiple cloning site”. In certain embodiments of the invention, it may be desirable to include vector fragments containing such elements in linkage reactions. However, in many embodiments, it will be desirable to create fragments and/or hybrid molecules without employing the use of restriction enzymes. As techniques for such restriction-free nucleic acid manipulation become more accepted, the need for polylinkers in inventive vectors and reactions will diminish.

Other Elements

Those of ordinary skill in the art will readily appreciate that any of a variety of other sequence elements may be included in vector fragments according to the present invention. The foregoing has been intended to provide merely a sampling of certain examples of sequence elements that are currently commonly found in vector sequences. One of the advantages of the present invention is that, by providing techniques and reagents that allow the ready production of specifically designed vectors through the assembly of prepared fragments, it is expected that the invention will also help researchers expand the range of sequence elements utilized in vector applications.

Insert Elements

As will be apparent to those of ordinary skill in the art, any nucleic acid sequence may be employed as an insert element according to the present invention. A researcher may choose any sequence or sequences s/he likes to be linked to vector sequences. Also, more than one insert element may be employed. Furthermore, each insert element may be provided as a single insert fragment, or may be distributed over multiple insert fragments that will be linked together in series in the final hybrid product. In certain embodiments of the invention, part or all of a given insert element may even be prepared as a single fragment that also includes part or all of one or more vector elements. Any collection of contiguous insert sequences is considered a single insert element for the purpose of the present invention.

Those of ordinary skill in the art will recognize that the classification of particular sequences as “insert elements” as compared with “vector elements” is not critical to the invention. In fact, the inventive recognition that a “vector” need not be a single discrete molecular entity in a sense renders such distinctions arbitrary. Nonetheless, both the concept and the terminology of a “vector backbone” and an “insert” are well established in molecular biology and therefore can be useful for the purposes of clarity and communication.

Preparation and Linkage of Fragments

In general, any method may be used to prepare fragments for hybridization and/or linkage according to the present invention. However, it is preferred that, for each hybrid molecule to be assembled, at least one fragment is prepared without the use of restriction enzymes, and preferably without the use of any endonuclease.

In certain preferred embodiments of the invention, fragments are prepared in a form that allows them to be linked together by ligation. In other embodiments, fragments are prepared in a manner that allows them to be linked together by splicing. In particular, U.S. patent applications Ser. No. 08/814,412, U.S. Ser. No. 09/399,593, U.S. Ser. No. 09/225,990, and PCT/US00/0189, and U.S. Pat. Nos. 5,498,531 and 5,780,272, each of which is incorporated herein by reference, contain thorough descriptions of methods and strategies useful in the preparation of nucleic acid (RNA or DNA) fragments that contain flanking intronic sequences and can be linked to one another by trans- or cis-splicing. In yet other embodiments, fragments are prepared in a form that allows topoisomerase-mediated linkage.

Often, it will be desirable to prepare fragments so that, for each linkage reaction to be performed in the assembly of a hybrid molecule, the fragments are designed to associate with one another in only one way and to produce only a single major linkage product. For example, fragments may be prepared so that each has single-stranded overhangs on one or both ends, and only fragments that are to be adjacent to one another in a hybrid molecule have complementary overhangs. Alternatively or additionally, fragments may be engineered to include intronic elements that are only compatible with the intronic elements on adjacent fragments. Such “directed linkage” (i.e., linkage in only one arrangement) of fragments discussed above is particularly desirable where multiple fragments (i.e., three or more, preferably four or more, and more preferably five or more) are to be linked together in a single linkage reaction. For linkage reactions containing small numbers of fragments (2 or 3), directed linkage can be assured by controlling the phosphorylation state of the relevant fragment ends.

In other preferred embodiments of the invention, it may be desirable to prepare fragments so that they can become linked to one another in any of a variety of different ways. This phenomenon is referred to herein as “linkage degeneracy”. In such embodiments, a single linkage reaction can generate a “library” of different hybrid molecules that can subsequently be distinguished and/or separated from one another as desired.

In yet other preferred embodiments of the invention, fragments can be designed for directed ligation as described above, but then multiple alternative versions of each particular fragment can be provided in the same linkage reaction so that, once again, a library of hybrid molecules is produced in a single linkage reaction. This phenomenon is referred to herein as “selection degeneracy”. For example, fragments A, B, and C can be designed and prepared so that they can only be linked to one another in the arrangement ABC (which can be a linear or a circular arrangement). If multiple different A fragments (e.g., A1, A2, A3, . . . An), multiple different B fragments, and/or multiple different C fragments are employed in a single linkage reaction, then a library of different hybrid molecules, each having an ABC structure, will be produced in that reaction (e.g., A1B17C3, A1B1C1, A1B2C1, etc.). Those of ordinary skill in the art will readily appreciate that the different versions of the A fragment need not bear any relationship to one another other than being designed to be link only to a B fragment, etc. Alternatively, each version of a given fragment could, for example, contain different varieties of the same vector element(s) or element portion(s) (e.g., different drug resistance genes).

Still other preferred embodiments combine the two kinds of degeneracy discussed above, so that a single linkage reaction may create a library of hybrid molecules in which both the arrangement and selection of fragments is varied.

According to the present invention, particularly preferred fragments for use in accordance with the present invention contain one or more single-stranded overhangs available for hybridization with complementary overhangs on other fragments. It is most preferred that such overhang-containing fragments be prepared without the use of restriction enzymes. It is particularly preferred that such fragments be prepared using RNA-Overhang Cloning (ROC) or DNA-Overhang Cloning (DOC), as described for example in U.S. Ser. No. 09/225,990; PCT US00/00189; and U.S. Ser. No. 09/478,263, each of which is incorporated herein by reference in its entirety (see also Examples 5-8).

Once a hybrid molecule is created by hybridization or linkage of vector and/or insert fragments, it may be replicated by any available in vitro or in vivo mechanism. In certain preferred embodiments of the invention, hybridization or linkage reactions themselves, or isolated or purified hybrids prepared from such reactions (e.g., by gel electrophoresis), may be directly transformed or transfected into host cells (or otherwise introduced into a host system). In some cases, it may be desirable to perform one or more manipulations prior to introducing a hybrid molecule into a host cell. For example, linkage of two fragments created using some embodiments the ROC methodology will produce a hybrid molecule that includes some regions of double-stranded RNA that may not be stable inside certain host cells. Accordingly, it may be desirable to perform at least a single round of DNA replication of such a hybrid prior to introducing it into a cell.

Other circumstances in which such additional manipulations (e.g., nick repair, etc.) are desirable will be apparent to one of ordinary skill in the art.

Kits

As discussed herein, one aspect of the present invention comprises the recognition that vectors are comprised of modular elements and can be assembled from individually prepared fragments. One part of this recognition includes the realization that vector fragments can be provided as isolated cassettes, ready to be assembled by a user or a manufacturer.

In one embodiment of the invention, a variety of different possible vector elements is offered to a user who selects particular pieces of interest. Fragments that together comprise these pieces are then prepared and are provided to the user for assembly into a vector. Optionally, reagents for performing the assembly (e.g., ligase if the fragments are prepared with overhangs amenable to linkage by ligation; splicing reagents if the fragments are prepared for linkage by splicing; etc.). Alternatively, the fragments may be linked together into a “designer vector” before being provided to the user.

In other embodiments of the invention, kits are provided that contain multiple optional fragments, each of which contains a selected vector element or elements, or fragment(s) thereof, so that a user can readily assemble any of a variety of different vectors my mixing different collections of fragments together in linkage reactions. For example, a bacterial expression vector kit could be provided that contains (a) a first collection of first fragments, each of which contains the pTac promoter and also contains a portion of an antibiotic resistance gene, where different fragments in the collection contain portions of different antibiotic resistance genes; (b) a second collection of second fragments, each of which contains the remainder of one of the antibiotic resistance genes and also contains the ColE1 origin of replication; and (c) ligation reagents. A user could then select particular first and second fragments that, when ligated with his or her chosen insert fragment(s), would create a hybrid containing a chosen antibiotic resistance gene and the insert element under control of the pTac promoter. Those of ordinary skill in the art will immediately recognize the infinite variety of related other kits that could alternatively or additionally be provided.

The inventive recognition of vector modularity also provides a new perspective on valuable reagents, and systems for providing such reagents to users. For example, in addition to kits as discussed above, reagent providers could prepare catalogs or menus (either paper or electronic) from which users can select particular desired vector elements or fragments. In certain preferred embodiments of the invention, the catalog or menu is presented on a World Wide Web site that the user can access and through which the user can place an order. In other embodiments a paper form is provided, or information about telephone contact is provided. As discussed above, selected fragments may be provided to the user as isolated fragments, as fragment collections, as linked pieces (e.g., complete vectors), as kits (e.g., including linkage reagents, purification reagents, amplification reagents, instructions for use and/or other relevant materials), or in any other desirable form. The invention therefore provides, in addition to the various techniques and reagents discussed herein, new methods of doing business in the area of molecular biology reagents.

Hybrid Molecules

As discussed herein, the techniques and reagents provided by the present invention allow the ready assembly of any of a variety of hybrid molecules, generated by hyrbidization and/or linkage of vector and/or insert fragments. In some embodiments of the invention, a vector is assembled from vector fragments (via one or more than one linkage reactions) prior to linkage of vector sequences with insert sequences. In other embodiments, assembly of the complete, final hybrid product is accomplished in a single linkage reaction. In other embodiments, one or more linkage fragments is/are linked to one or more vector fragments in a first linkage reaction, and one or more additional linkage reactions are subsequently performed to add additional vector and/or insert fragments. Each and every hybrid molecule produced in such a linkage reaction is encompassed within the scope of the present invention.

### EXAMPLES

### Example 1

### Assembly of a λ Vector/Insert Hybrid

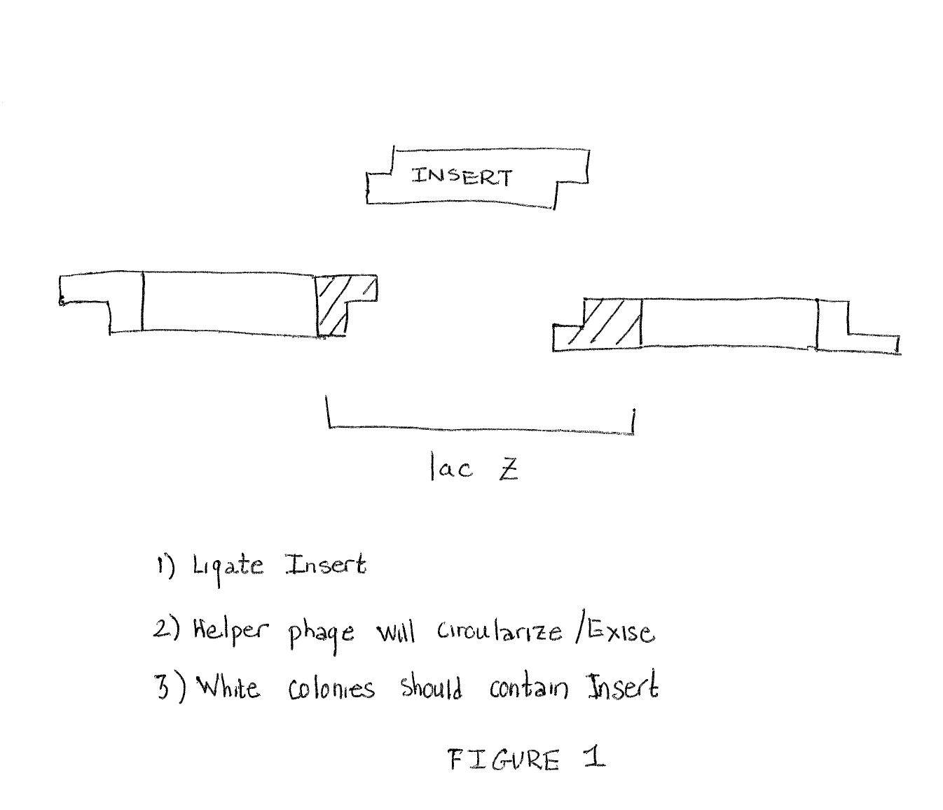

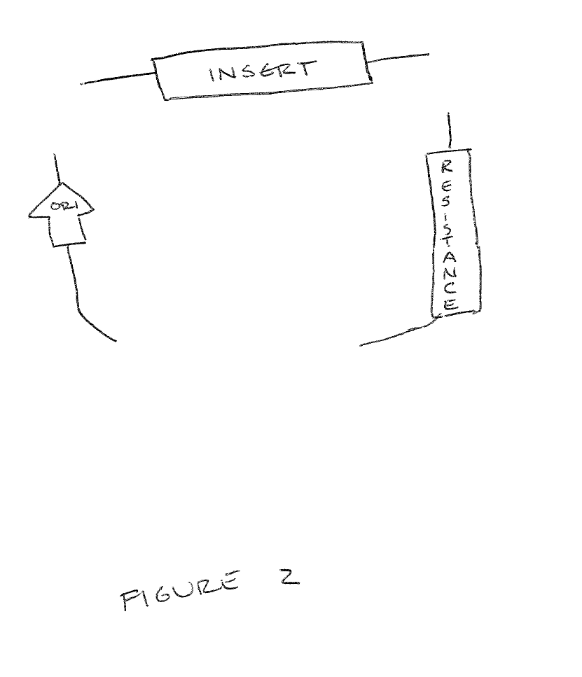

FIG. 1 presents an inventive reaction for the assembly of a hybrid molecule containing two λ phage arms (a λ cloning vector) separated by a chosen insert. As is well known, λ vectors are particularly useful for the cloning of relatively large (up to about 50 kB) fragments. The insert-containing hybrids can be packaged (typically through the use of helper phages) into phage heads in vitro. Although the efficiency of packaging can be relatively low (around 10%), the subsequent efficiency of genome transfer into bacteria through infection is close to 100% (see, for example, Ausubel et al., Current Protocols in Molecular Biology, Unit 1.10, Current Protocols, 1987, the entire contents of which are incorporated herein by reference).

### Example 2

### Assembly of a Bacterial Vector/Insert Hybrid

FIG. 2 presents an inventive reaction for the assembly of a hybrid molecule containing a bacterial origin of replication, an antibiotic resistance gene, and a chosen insert. The hybrid molecule is assembled by linkage of three fragments, each of which contains a single element. Preferably, the fragments are prepared to have complementary overhangs selected to provide for directional ligation. Alternatively, the indicated element in each fragment may be flanked by intronic components that direct appropriate trans-splicing reactions in vivo or in vitro.

### Example 3

### Assembly of a Bacterial Vector/Insert Hybrid in Which the Insert Disrupts a Detectable Element

The inventive reaction depicted in FIG. 3 differs from that shown in FIG. 2 (and discussed above in Example 2) in at least two ways. First, the two vector fragments employed in the reaction of FIG. 3 each contain a part of the bacterial origin of replication, so that only hybrid molecules in which these two fragments are properly linked together will be able to replicate in bacteria. Also, each vector fragment contains a portion of the ampicillin resistance gene. If a hybrid is assembled that does not include an insert, the ampicillin resistance gene will be re-created (unless some mutation occurs) and bacteria containing the resulting hybrid will be resistant to both tetracycline and ampicillin. By contrast, the ampicillin gene will not be re-created in hybrid that do contain the insert. Thus, bacteria containing complete hybrids will be distinguishable from those containing partial hybrids that lack insert because those containing complete hybrids will be resistant to tetracycline but not ampicillin, whereas those containing partial hybrids will be resistant to both.

The strategy depicted in FIG. 3 is particularly useful for fragments that do not have directionally specific ends. For example, if blunt-ended fragments are to be employed, or if the both ends of the insert fragment (and both ampicillin fragment ends) contain identical overhangs, the ability to identify desirable hybrids from the universe of possible hybrids is particularly useful.

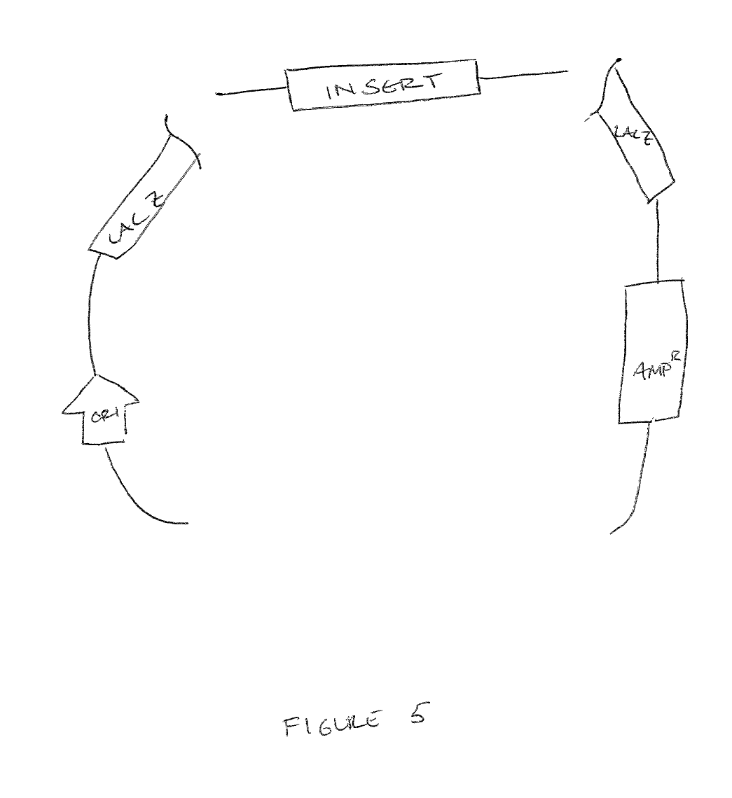

The strategy depicted in FIG. 4 is analogous to that depicted in FIG. 3 except that hybrids containing insert are distinguishable from those lacking insert on the basis of a blue/white screen rather than a growth/no growth screen.

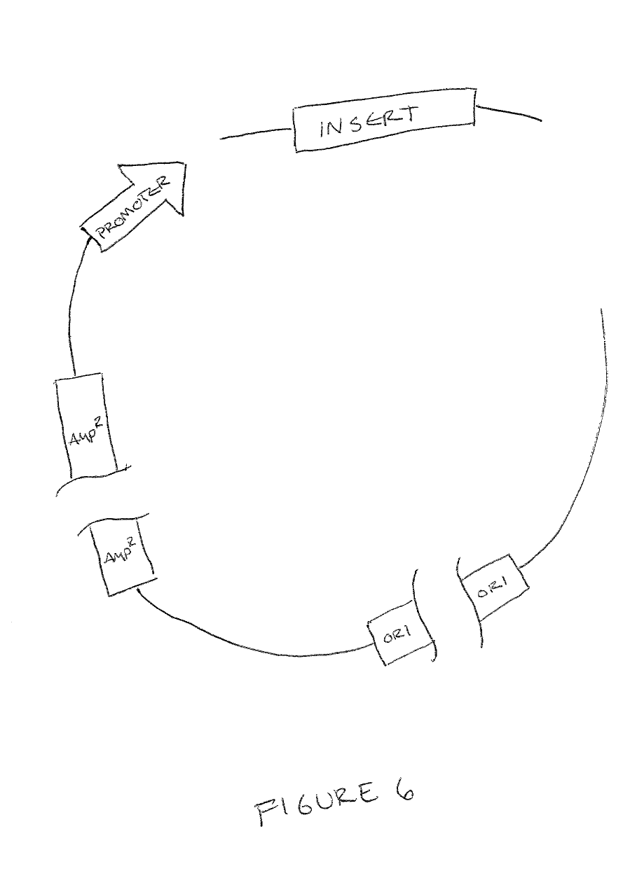

The strategy depicted in FIG. 5 is also similar, except that linkage of the vector fragments is not required to create a functional origin of replication. For this strategy, it is generally preferred that at least the vector fragments be engineered for directional linkage, so that they can only be linked to one another in a single orientation.

### Example 4

### Assembly of a Hybrid Bacterial Expression Vector/Insert Construct by 4-Way Ligation

The inventive strategy depicted in FIG. 6 shows simultaneous linkage of three different vector fragments with an insert fragment. A hybrid vector molecule containing both an origin of replication and an ampicillin resistance gene can only be assembled through proper linkage of the three vector fragments. Thus, selection strategies can be employed to identify desirable hybrid molecules. Such molecules can then be screened for expression of the insert in order to identify those that are complete as compared with those that contain only vector sequences.

### Example 5

### Assembly of a Hybrid Bacterial Vector/Insert Molecule Using DOC

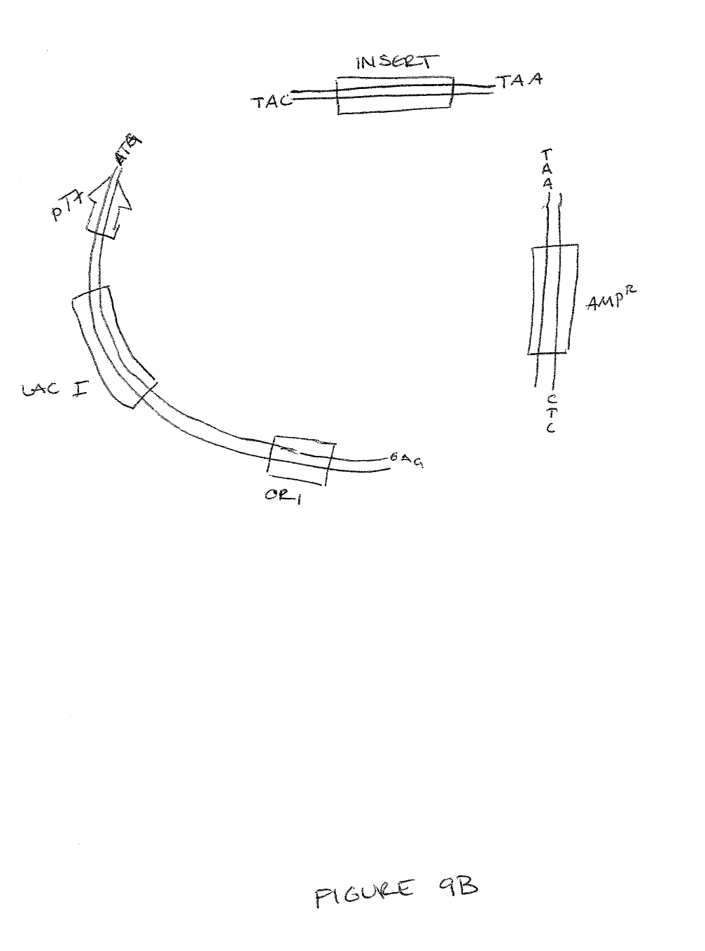

The inventive scheme depicted in FIG. 9 was carried out as follows. Vector fragments were amplified from the pET 19b vector (Novagen, Madison, Wis.) using the following primers (lower case letters indicate RNA residues; upper case letters indicate DNA residues):

EV-1 (5′-cauGGTATATCTCCTTCTTAAAG; SEQ ID NO:1),
EV-2 (5′-cucATGACCAAAATCCCTTAAC; SEQ ID NO:2),
EV-3 (5′-gagATTATCAAAAAGGATCTTC; SEQ ID NO:3),
and
EV-4 (5′-uaaCTAGCATAACCCCTTGG; SEQ ID NO:4).

Were used together to generate vector fragment 1, containing the bacterial origin of replication, the LacI gene, and the pT7 promoter; EV-3 and EV-4 were used together to generate vector fragment 2, containing the Amp gene.

In a separate DOC reaction, an insert fragment containing the Lac Z gene was amplified from the pBluescript II SK (−) vector (Stratagene, La Jolla, Calif.), with primers

5′ Lac Z (5′-augACCATGATTACGCCAACG; SEQ ID NO:5)
and
3′ Lac Z (5′-uuaCAATTTCCATTCGCCATTC; SEQ ID NO:6).

100 μl PCR Reactions contained 5 ng of template DNA, 1× cloned PFU buffer (Stratagene, La Jolla, Calif.), 1 mM MgSO₄, 200 μM of each dNTP, 1.45 U cloned PFU (Stratagene), 1.25 U PFU Turbo™ polymerase and 50 pM of each primer. Reactions were performed in a Robocycler (Stratagene, La Jolla, Calif.) as follows: 1 cycle 95° C., 5 min; 53° C., 3 min; 72° C., 6 min (10 min for vector fragment 1); 30 cycles, 95° C.; 1 min; 53° C., 1 min; 72° C., 3 min (8 min for vector fragment 1); and 1 cycle 72° C. 10 min.

Products of the PCR reactions were separated on a 1% agarose gel, and purified using the GENECLEAN II kit (Vista, Calif.). 12 μl of each purified fragment was placed separately in 1× first strand buffer (Life Technologies, Rockville, Md.) with 10 mM DTT, 5 mM of each dNTP, and 200 U M-MLV (Life Technologies). Reactions were incubated for 20 min at 42° C. Reactions were then placed at 70° C. for 10 min to heat kill the enzyme.

Primer ribonucleotides were removed from the PCR products by hydrolysis with NaOH. 6 μL of 1 N NaOH were added to each reaction, and the mixtures were incubated for 30 min at 45° C. 6 μl of 1 N HCL, 4 μl of 10× ligase buffer (USB, Cleveland, Ohio), and 10 U of T4 PNK (USB) were then added. Reactions were incubated at 37° C. for 30 min. Phosphorylated fragments were combined in equimolar amounts (approximately 50 ng) and ligated with 10 U of T4 DNA ligase (USB) at 25° C. for 2 hrs. 5 μl of the ligation reaction was then transformed into E. coli.

### Example 6

### Assembly of Hybrid Vector/Insert Molecules Using ROC with Internal Terminators

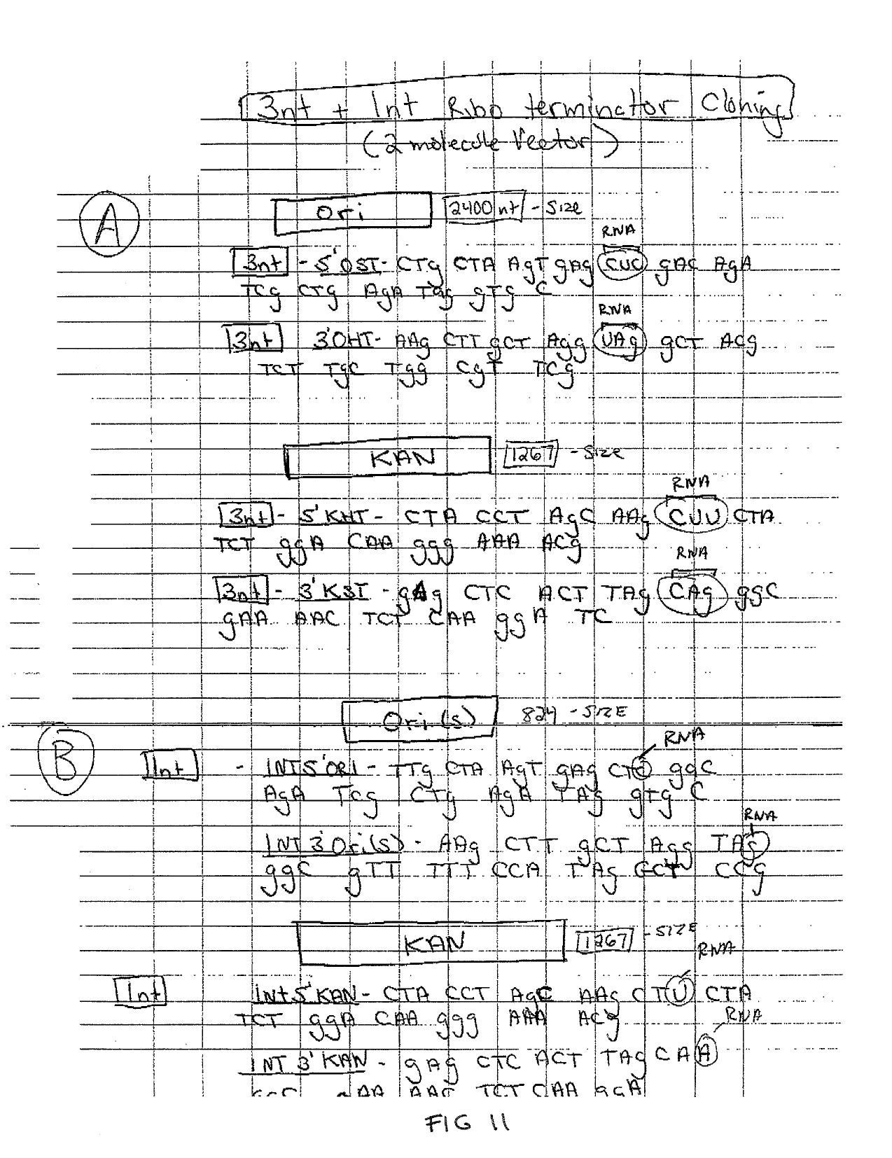

We prepared hybrid vectors containing an origin of replication (Ori) fragment and a kanamycin resistance gene (KAN) fragment, by amplifying each fragment with primers that contained one or more residues not copied by the DNA polymerase utilized in the reaction (i.e., “terminator” residues). The Ori fragment was amplified from pET19b (Novagen, Madison, Wis.); the KAN fragment was amplified from pCR 2.1 (Invitrogen, Carlsbad, Calif.). FIG. 11 shows the various primers used and fragments generated. As will be seen, some reactions generated a 2400 bp ori fragment; others generated an 824 bp fragment, (denoted “Ori(s)” because it is smaller). The smaller fragment, Ori(s), lacks an 11 pb direct repeat that can create a deletion hotspot when it is present.

PCR reaction cycling, product annealing and E. Coli transformation were performed as described in Examples 7 and 8.

### Example 7

### Assembly of a Hybrid Vector/Insert Molecule Using ROC with Single Nucleotide Terminators

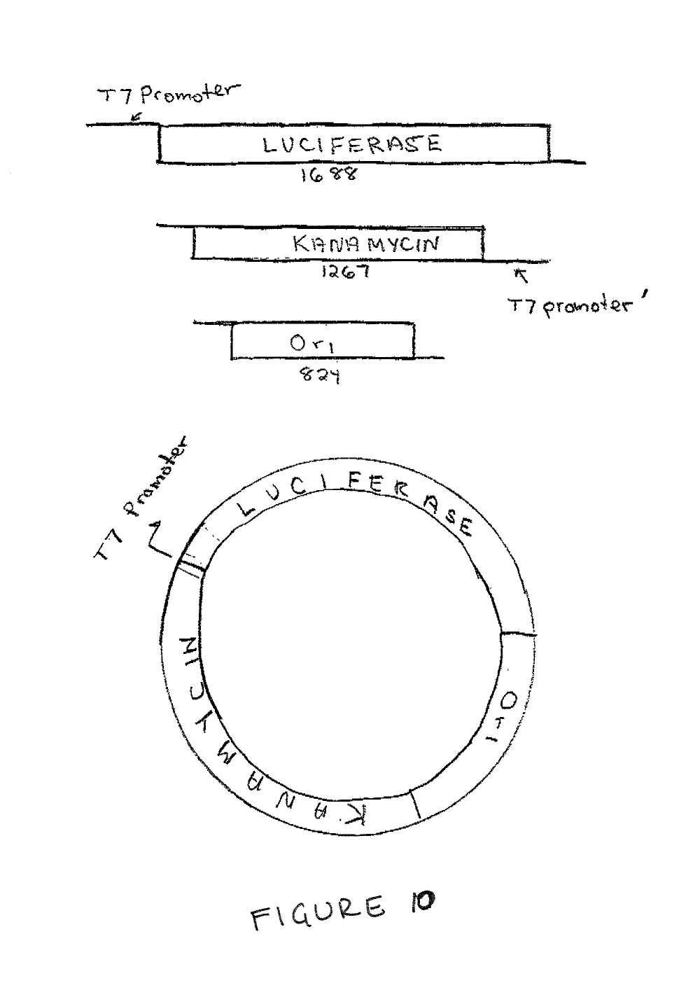

The vector/insert hybrid molecule depicted in FIG. 10 was generated as follows. The ori-containing vector fragment was amplified from pET 19b (Novagen, Madison, Wis.) using primers (lower case letters indicate RNA residues; upper case letters indicate DNA residues)

5′OST (5′-CTGCTAAGTGAGcucGACAGATCGCTGAGATAGGTGC; SEQ ID NO:5) and
1N3′Ori(s)(5′-AAGCTTGCTAAGTAgGGCGTTTTTCCATAGGCTCCG; SEQ ID NO:6)

The vector fragment containing the Kanamycin resistance gene was amplified from pCR2.1 Topo (Invitrogen, Carlsbad, Calif.) using primers

1NT5′KAN (5′CTACCTAGCAAGCTuCTATCTGGACAAGGGAAAACG; SEQ ID NO:7) and
T7 3′KAN (5′CCCTATAGTGAGTCGTATTAaGGCGAAAACTCTCAAGGATC; SEQ ID NO:8).

The insert fragment containing the luciferase gene was amplified from pGI II basic (Promega, Wis.) using primers

tCS1 (5′TTAATACGACTCACTATAGGGATGGAAGACGCCAAAAACATA; SEQ ID NO:9) and
tCS8 (5′-GAGCTCACTTAGCAGTTACAATTTGGACTTTCCGCC; SEQ ID NO:10).

Each 100 μl reaction contained 50 pM of each primer, 1× cloned Pfu Buffer (10 mM (NHy)₂SO₄, 20 mM Tris (pH 8.8), 2 mM Mg SO₄, 10 mM KCE, 0.1% Triten x-100 and 0.1 mg/me Bovine serum Albumin), 1 mM additional mg SO₄, 0.3 mM each dNTP, 5-10 ng template DNA and 1.25-1.85 units of both cloned Pfu and Pfu Turbo polymerases (strategies, La Jolla, Calif.). The Ori fragment was amplified in a reaction involving (1) one cycle of 95° for 3 min; 46-60° for 2 min; (2) 35 cycles of 95° for 30 sec; 48-60° for 30 sec; and 72° for 3 min; and (3) one cycle of 95° for 30 sec; 48-60° for 30 sec; and 72° for 8 min. The KAN and LUC fragments were amplified in similar reactions except that the 35 cycles contained a 4.5 min 72° step.

PCR products generated in these reactions were gel purified using the Qiaquick gel extraction kit (Qiagen, Valencia, Calif.). Approximately 80 ng of each fragment was combined in a 20 μl reaction volume. Two (2) μl 10× USB ligation Buffer (660 mM Tris-HCL (pH7.6), 66 mM MgCl₄, 100 mM DTT, 660 μM ATP) (USB, Cleveland, Ohio) was then added, to make a 1× reaction mix. The reaction was heated to 65° C. for 8 minutes, and then slow cooled for 20 minutes (to 35-40° C.) to allow the fragments to anneal. Samples were spun and allowed incubate another 15 minutes at room temperature.

The annealing reaction was precipitated by adding 100 μl of 100% ethanol, followed by a 15 minute incubation at −80° C., and a 70% and 100% wash. Electrocompetent DH5α cells were transformed using a Biorad E. coli pulser (Biorad, Hercules, Calif.). Five (5) μl of each annealing reaction was combined with 40 μl of Elexctromax DH5α-E cells (Life technologies, Rockville, Md.) Individual clones generated in this experiment were isolated, restriction mapped, and sequenced; all junctions were correct.

Those of ordinary skill in the art will appreciate that, as with Example 6, the ROC technique described in this Example utilizes primers containing internal ribonucleotide residues (in one case, 3 residues were used; in other cases only one) flanked by DNA residues. The overhangs created in these ROC PCR reactions, therefore, have only a single “ribo” residue; other overhang residues are DNA. In separate experiments, we have demonstrated that any individual ribonucleotide (i.e., rA, rG, rU, or rC) can act effectively to block extension of a complimentary strand by an appropriate DNA polymerase, so that overhangs are created (see, for example, Example 6). We have also owed that single 3′-Omethyl residues are similarly effective. Primers containing 3′-Omethyl residues can often be synthesized more easily (e.g., due to higher coupling efficiencies) than those containing inbanucleotides, and will generally be more stable, so that they are preferred for many applications.

### Example 8

### Streamlined Cloning

Inventive modular vector fragments may be prepared, annealed together, and transformed into host cells, without enzymatic ligation. For example, we assembled a two-fragment vector, by preparing one fragment (KAN) containing the kanamycin resistance gene, and one fragment (Ori) containing an origin of replication.

Specifically, two 100 μl PCR reactions were performed to amplify each of the two components of the vector. Each reaction contained 50 pM of each primer, 1× Cloned Pfu Buffer (10 mM (NH₄)₂SO_{4,} 20 mM Tris (pH 8.8), 2 mM MgSO₄, 10 mM KCl, 0.1% Triton X-100 and 0.1 mg/ml bovine serum albumin), 1 mM additional MgSO4, 0.3 mM of each dNTP, 5-10 ng of plasmid template and 1.25-1.85 units of both cloned Pfu and Pfu Turbo polymerases (Stratagene, La Jolla, Calif.).

The following chimeric RNA/DNA primers were purchased from Oligo's Etc.( Willsonville, Oreg.): (ribonucleotides are in lower case)

| | |
|---|---|
| 1NT 5′KAN-CTACCTAGCAAGCTuCTATCTGGACAAGGGAAAACG | (SEQ ID NO:11) |
| 1NT 3′KAN-GAGCTCACTTAGCAaGGCGAAAACTCTCAAGGA | (SEQ ID NO:12) |
| 1NT5′Ori-TTGCTAAGTGAGCUcGACAGATCGCTGAGATAGGTGC | (SEQ ID NO:13) |
| 1N3′Ori(s)-AAGCTTGCTAAGTAgGGCGTTTTTCCATAGGCTCCG | (SEQ ID NO:14). |

Primers INT 5′KAN and INT 3′KAN were used to amplify the Kan fragment from pCR 2.1 Topo (Invitrogen, Carlsbad, Calif.). Primers 1NT5′Ori and 1N3′ Ori(s) were used to amplify the Ori fragment from pET 19b (Novagen, Madison, Wis.). The following cycles were performed: one cycle of 95°, 3′, 48-60°, 2′, 72°, 8′; followed by 35 cycles of 95°, 30 sec, 48-60°, 30 sec, 72°, 3′ for Ori fragment, 4.5′ for Kan and Luc fragments. A final cycle with an 8′ 72° step was performed in all cases.

Approximately 80 ng of each fragment (5 μl each) produced in the PCR reactions was combined and mixed. 5 μl of this reaction was then transformed into 100 μl of chemically competent DH5α cells. Positive clones were isolated and mapped; sequence junctions appear to be correct.

### Other Embodiments

Those of ordinary skill in the art will appreciate that the foregoing represents certain preferred embodiments of the invention, but is not intended to limit the spirit or scope of the following claims.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

1. A method of preparing a vector, the method comprising steps of:
providing at least two isolated nucleic acid molecules, each of which contains a portion of vector sequence;
providing at least one isolated nucleic acid molecules containing insert sequence; and
admixing the nucleic acid molecules with one another under linkage conditions so that a hybrid molecule in which each of the isolated molecules is linked together is produced.

2. The method of claim 1 wherein:
the isolated nucleic acid molecules each contain at least one overhang that is complementary with an overhang on at least one of the other molecules; and
the step of admixing comprises admixing under ligation conditions.

3. The method of claim 1 wherein:
the isolated nucleic acid molecules each contain at least one intronic element that is characterized by an ability to trans-splice with a compatible intronic element on at least one of the other molecules, and
the step of admixing comprises admixing under ligation conditions.

4. The method of any one of claims 1-3, further comprising a step of:
introducing the hybrid molecule into a cell.

5. The method of claim 1 wherein each of the isolated vector molecules contains at least a portion of a vector element selected from the group consisting of replication elements, vector detection elements, expression elements, gene fusion elements, protein fusion elements, polylinker elements, and combinations thereof.

6. A hybrid molecule assembled according to the method of claim 1.

7. A collection of vector fragments, each of which contains at least a portion of a vector element selected from the group consisting of replication elements, vector detection elements, expression elements, gene fusion elements, protein fusion elements, polylinker elements, and combinations thereof.

8. A method of providing biotechnology reagents, the method comprising steps of:
providing a menu of vector fragments, each of which contains at least a portion of a vector element selected from the group consisting of replication elements, vector detection elements, expression elements, gene fusion elements, protein fusion elements, polylinker elements, and combinations thereof,
receiving from a user a request for at least one vector fragment; and
providing the requested vector fragment to the user.

9. The method of claim 8, wherein:
the step of providing a menu comprises providing a World Wide Web at which the user may enter selections.

10. The method of claim 8 or claim 9, wherein:
the step of receiving comprises receiving a request for at least two vector fragments; and
the step of providing comprises:
linking the requested vector fragments to one another as a hybrid molecule; and
providing the hybrid molecule to the user.

11. A method of preparing a vector, the method comprising steps of:
providing at least two isolated nucleic acid molecules, each of which contains a portion of vector sequence and each of which comprises a single-stranded portion at a terminus thereof, at least two such single-stranded portions being complimentary to one another; and
admixing the nucleic acid molecules with one another under conditions that allow hybridization of the complementary single-stranded portions.
